(19) United States
(12) Patent Application Publication
(10) Pub. No.: US 2019/0298261 A1
(43) Pub. Date: Oct. 3, 2019

(54) HEART ACTIVITY MONITORING SYSTEM WITH V-POTENTIAL SENSORS

(71) Applicant: Heart.Zone Limited Liability Company, Moscow (RU)

(72) Inventor: Vladimir Savchenko, Moscow (RU)

(21) Appl. No.: 16/302,789

(22) PCT Filed: May 16, 2017
(86) PCT No.: PCT/RU2017/000313

§ 371 Date: Nov. 19, 2018

Related U.S. Application Data
(60) Provisional application No. 62/337,856, filed on May 17, 2016.

(51) Int. Cl. A61B 5/00; A61B 5/0408; A61B 5/04; A61B 5/024

(52) U.S. Cl. CPC A61B 5/6803; A61B 5/04085; A61B 5/04012; A61B 5/6817; A61B 5/0024; A61B 5/02405; A61B 5/0006

(57) ABSTRACT

The invented heart activity sensory system, commonly called a heart monitor, consists of at least two skin contact sensors which can be attached to or integrated with wearable devices or accessories such as headsets, glasses, goggles, hats, helmets, necklaces, pendulums, garments, or directly to a body. Each sensor has an electrical electrode to establish a contact with skin for the purpose of measuring skin voltage potential and heart activity using the electrocardiogram (ECG or EKG) method. The sensors can communicate wirelessly with each other, calculate heart activity characteristics, record the characteristics in a memory, and inform the user visually, audibly, or with tactile means about the user's heart activity. The sensors can also transmit these characteristics wirelessly to a host device such as a smartphone.

### TECHNICAL FIELD

This disclosure generally relates to portable heart activity monitors, portable heart rate detection systems, portable electrocardiography systems, and the like devices. More particularly, this disclosure relates to an apparatus which integrates skin voltage potential sensors with wearable devices and accessories and establishes a heart activity measuring system.

### DESCRIPTION OF RELATED ART

Athletic activity is an important factor for many individuals in maintaining a healthy lifestyle. It was found to be advantageous to track and record heart activity data especially during a physical exercise. Also, continuously tracking heart activity helps people to better gauge their health and detect heart abnormalities early for timely treatment. There are known many devices designed to monitor human heart activity. For example, a common device is a heart rate monitor including a chest belt having two electrical contacts which should contact the chest of a user and enable the heart rate monitor to measure a human heart rate. This type of heart rate monitor is found to be inconvenient to use by many individuals.

Other heart rate monitors involve an optical or light-based technology. These hart rate monitors typically include a light source and a light detector. A light is shined from the light source, directed through skin of the user to a blood vessel, and the reflected light sensed by the light detector. The heart rate monitor further measures a blood motion within a vessel of the user to determine heart activity. The light-based heart rate monitors are typically integrated into wearable accessories such as watches, wrist bands, arm bands, and the like. One well known drawback of the light-based heart rate monitors includes inconsistency in heart rate measurement, especially when the user is in motion.

Electrocardiography devices have been proven to be more reliable but they are typically bulky, non-portable, or require an extra device tightly attached to a chest of the user. The electrocardiography devices were also found to be inconvenient to use. Accordingly, there is still a need in the art to improve heart activity monitors, make them more reliable, user friendly, and integrated with existing wearable devices, fashion accessories, and garments.

### SUMMARY

This section is provided to introduce aspects of embodiments of this disclosure in a simplified form that are further described below in the section of Detailed Description of Example Embodiments. This summary is not intended to identify key features or essential features of the claimed subject matter, nor is it intended to be used as an aid in determining the scope of the claimed subject matter. The aspects of embodiments of this disclosure are designed to overcome at least some drawbacks existing known in the art.

According to one aspect of this disclosure, there is provided an apparatus for heart activity monitoring. The heart monitoring apparatus or sensory system consists of at least two sensors which can be integrated into or attached to wearable devices, fashion accessories and garment worn by a human. The heart monitoring apparatus uses voltage potential measured at skin surface using an electrocardiogram (ECG or EKG) method with at least two sensors with electrodes attached to skin at different locations on the user's body.

The wearable devices and accessories which can be integrated with the heart monitoring voltage potential or V-potential sensor include but are not limited to: headsets with in-ear headphone, bone-conducting headphones attached behind the ear or in front of the ear and a single-ear headphone, glasses, goggles, mask, visor, helmet, hat, hood, headband, bandana, and a swimming cap. The garments which can be integrated with the heart monitoring sensor include but are not limited to: shirts, jerseys, shorts, bras, cycling or swimming suits, sport bibs, and body tights.

The heart monitoring sensory system consist of the first sensor with an electrode attached to skin in one part of the human body and the second sensor with an electrode attached to skin in a different part of the human body. The sensors could be integrated into an existing device, accessory, or garment or be a stand-alone sensor device. Each sensor measures electrical voltage potential of the skin at a place of contact. The first sensor is configured as a supplier sensor which wirelessly transmits the measured skin voltage potential to the second sensor configured as a consumer sensor. The consumer sensor compares skin voltage potential from the supplier sensor with its own skin voltage potential and calculates heart activity characteristics such as the heart rate or heart beats per minute, heart variability rate, and heart rhythm using the ECG method. The consumer sensor then displays, stores, and wirelessly transmits calculated heart activity characteristics to another device such as a smartphone or any other monitoring device.

According to another example embodiment, a system for heart activity monitoring is provided. The system may include at least one first sensor positioned within at least one first area of a body of a user. The first sensor can be integrated in a first accessory worn by the user. The first sensor can be configured to sense at least one electrical signal from skin of the user within the at least one first area of the body of the user. The electrical signal may be indicative of the heart activity of the user. The system may further include a second sensor positioned within a second area of the body of the user. The second sensor can be integrated in a second accessory worn by the user. The second sensor can be configured to sense a second electrical signal from the skin of the user within the second area of the body of the user. The second electrical signal can be indicative of the heart activity of the user. The second electrical signal can be sensed simultaneously with the electrical signal sensed by the first sensor. The second sensor can be further configured to wirelessly receive the electrical signal from the first sensor. The second sensor can be configured to perform a comparative analysis of the first sensor electrical signal and the second electrical signal to obtain heart activity data.

According to yet another example embodiment, a method of heart activity monitoring is provided. The method may include sensing, by at least one first sensor positioned within at least one first area of a body of a user, at least one electrical signal from skin of the user within the at least first area of the body of the user. The first sensor can be integrated with a first accessory worn by the user. The electrical signal can be indicative of the heart activity of the user. The method may further include sensing, by a second sensor positioned within a second area of the body of the user, a second electrical signal from the skin of the user within the second area of the body of the user. The second sensor can be integrated with a second accessory worn by the user. The second electrical signal can be indicative of the heart activity of the user and can be sensed simultaneously with the at least one electrical signal. The method may include wirelessly receiving, by the second sensor, the electrical signal from the first sensor. The method may include performing, by the second sensor, a comparative analysis of the electrical signal and the second electrical signal to obtain heart activity data. The method may further allow wirelessly transmitting, by the second sensor, the heart activity data to a host device configured to wirelessly receive the heart activity data.

The details of the heart monitoring sensory system will be understood by a person with ordinary skills in the art from the drawings. Additional objects, advantages, and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 illustrates a block diagram of the heart monitoring apparatus;

FIG. 2A illustrates one embodiment of the heart monitoring sensor integrated with a wireless stereo headset;

FIG. 2B illustrates another embodiment of the heart monitoring sensor integrated with a wireless single ear headset;

FIG. 3A shows one embodiment of the invented sensor integrated with a watch;

FIG. 3B shows another one embodiment of the invented sensor integrated in an enclosure for wearing as pendulum on a necklace;

FIG. 3C is a view illustrating one embodiment of the invented sensor integrated in a button like enclosure for wearing on a clothing;

FIG. 4A is a view illustrating one embodiment for wearing the invented heart monitoring sensory system;

FIG. 4B is a view illustrating one embodiment for wearing a headset with the invented heart monitoring sensory system;

FIG. 5A is a view illustrating one embodiment of the invented sensor integrated with glasses;

FIG. 5B is a view illustrating one embodiment of the invented sensor integrated with goggles;

FIG. 6A is a view illustrating another embodiment for wearing the invented heart monitoring sensory system;

FIG. 6B is a view illustrating one embodiment for wearing a hat with the invented heart monitoring sensory system;

FIG. 7A is a view illustrating data chart measured by the invented sensors placed on an arm and a leg;

FIG. 7B is a view illustrating data chart measured by the invented sensors placed on an arm and in an ear;

FIG. 8 shows a flow diagram of a method for supplier sensor;

FIG. 9 shows a flow diagram of a method for consumer sensor;

### DETAILED DESCRIPTION OF EXAMPLE EMBODIMENTS

Introduction

The following detailed description of embodiments includes references to the accompanying drawings, which form a part of the detailed description. Approaches described in this section are not prior art to the claims and are not admitted to be prior art by inclusion in this section. The drawings show illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Aspects of embodiments will now be presented with reference to a headset or an apparatus for heart activity monitoring. These headset and apparatus may be implemented using electronic hardware, computer software, or any combination thereof. Whether such aspects of this disclosure are implemented as hardware or software depends upon the particular application and design constraints imposed on the overall system. By way of example, an element, or any portion of an element, or any combination of elements may be implemented with a “data processor” that includes one or more microprocessors, microcontrollers, Central Processing Units (CPUs), digital signal processors (DSPs), field programmable gate arrays (FPGAs), programmable logic devices (PLDs), state machines, gated logic, discrete hardware circuits, and other suitable hardware configured to perform various functions described throughout this disclosure. The data processor may execute software, firmware, or middleware (collectively referred to as “software”). The term “software” shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more embodiments, the functions described herein may be implemented in hardware, software, or any combination thereof. If implemented in software, the functions may be stored on or encoded as one or more instructions or code on a non-transitory computer-readable medium. Computer-readable media includes computer storage media. Storage media may be any available media that can be accessed by a computer. By way of example, and not limitation, such computer-readable media can comprise a random-access memory, a read-only memory, an electrically erasable programmable read-only memory, magnetic disk storage, solid state memory, or any other data storage devices, combinations of the aforementioned types of computer-readable media, or any other medium that can be used to store computer executable code in the form of instructions or data structures that can be accessed by a computer.

For purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

It should be also understood that the terms “first,” “second,” “third,” and so forth can be used herein to describe various elements. These terms are used to distinguish one element from another, but not to imply a required sequence of elements. For example, a first element can be termed a second element, and, similarly, a second element can be termed a first element, without departing from the scope of present teachings. Moreover, it shall be understood that when an element is referred to as being “on” or “connected” or “coupled” to another element, it can be directly on or connected or coupled to the other element or intervening elements can be present. In contrast, when an element is referred to as being “directly on” or “directly connected” or “directly coupled” to another element, there are no intervening elements present.

The term “V-potential sensor” shall be construed to mean voltage potential sensor measured at a surface of skin.

The term “host device” shall be construed to mean any computing or electronic device with data processing and data communication capabilities, including, but not limited to, a mobile device, cellular phone, mobile phone, smart phone, Internet phone, user equipment, mobile terminal, tablet computer, laptop computer, desktop computer, workstation, thin client, personal digital assistant, multimedia player, navigation system, game console, wearable computer, smart watch, entertainment system, infotainment system, vehicle computer, bicycle computer, or virtual reality device.

The term “earpiece” shall be construed to mean any device that can be placed into or near an outer ear of a user for purposes of outputting audio signals or noise reduction purposes. The term “earpiece” shall also mean any or all of the following, or shall be construed to mean that one or more of the following is an element of the “earpiece”: a headphone, an earbud, an earphone, an ear speaker, an ear pod, an ear insert, hearing aid device, and in-the-ear acoustic device.

The term “headset” shall be construed to mean a device that comprises at least one headphone only or a device that comprises at least one headphone and a microphone. Thus, a headset can be made with either a single-earpiece (mono) or a double-earpiece (mono to both ears or stereo). The term “headphones” is used herein to mean a pair of speakers or loudspeakers maintained close to a user's ears. Examples of headphones include circum-aural, supra-aural, earbud, and in-ear headphones. In-ear headphones are small headphones, sometimes also called earbuds, which are inserted in the ear canal or fitted in the outer ear. Although the embodiments of this disclosure are limited to wired headphones, those skilled in the art would appreciate that the same or similar embodiments can be implemented with wireless headphones or wireless headset.

The term “garment” shall be construed to mean any piece of clothing worn over skin of the user to cover the top part of the user. Some examples of a garment include a T-shirt, jersey, bra, cycling or swimming suits, sport bibs, body tights, or a pullover.

The term “heart activity” shall be construed to mean any vital, natural or biological activity of a human heart including heart beats or heart electrical activity. The term “heart activity signal” shall be construed to mean an analog signal characterizing a heart activity of a user. The term “heart activity data” shall be construed to mean any digital data characterizing a heart activity of a user. Some examples of heart activity signal include, but are not limited to, an ECG or EKG signal, heart activity wave signal, or heart activity impulse signal. Some examples of heart activity data include, but are not limited to, a heart rate, heartbeats per minute, a heart variability rate, a heart rhythm, an ECG or EKG represented in digital form, or any other vital or biometrical data associated with heart activity.

As outlined above, the aspects of embodiments of this disclosure provide an apparatus for heart activity monitoring. In other words, the embodiments of this disclosure integrate a heart activity voltage potential or V-potential measuring sensor into headphones or headset, a hat or a helmet, glasses, googles, a watch, a bracelet, a pendulum, a button, and other wearable accessories or garments. The heart activity V-potential measuring sensor is generally configured to sense, detect, or measure one or more heart activities of the user, generate heart activity data based, and transmit or cause transmitting the heart activity data to another sensor and a host device such as a smart phone or server for further processing, recording in a memory, or displaying.

The heart activity V-potential measuring sensor comprises at least one electrode which is configured to directly connect to skin of a user. In one embodiment, the first sensor with an electrode is placed inside an ear canal or fitted in an outer ear of the user, and establishes a reliable electrical contact with the skin. The second sensor with an electrode can be placed on a chest, arm, wrist, neck, shoulder, back, or leg of the user. In some implementations, the heart activity V-potential measuring sensor can be incorporated into a headset, a watch, or a band.

In another embodiment, the first electrode is placed inside a hat or a helmet and establishes a reliable electrical contact with the skin of a forehead. The second electrode can be placed on a chest or under an arm, in an armpit area, or on a neck, shoulder, or back of the user.

In yet another embodiment, the first heart activity V-potential sensor with an electrode is placed inside glasses on nose pads and temple frame and establishes a reliable electrical contact with the skin on a nose, temple, and behind an ear. The second heart activity V-potential measuring sensor with an electrode can be placed on a chest or on an arm, a wrist, or on a neck, shoulder, back, or a leg of the user.

In yet again another embodiment, the first heart activity V-potential measuring sensor with an electrode is placed inside goggles on eye pads and establishes a reliable electrical contact with the skin around an eye and on a temple. The second heart activity V-potential measuring sensor with an electrode can be placed on a chest or on an arm, a wrist, or on a neck, shoulder, back, or a leg of the user.

And in yet another embodiment, the first V-potential measuring sensor with an electrode is placed inside a pendulum hanging on a neck and establishes a reliable electrical contact with the skin on a chest. The second V-potential measuring sensor with an electrode can be placed on an arm, or a wrist, shoulder, back, or a leg of the user.

The heart activity measuring system can further comprise two or more sensors coupled with the electrodes and configured to measure electrical signals captured by electrodes and generated by heart muscles, filter the ECG signal, process this ECG signal, calculate various heart data from ECG signal, and transmit heart data to a host device.

Apparatus Architecture

Referring now to the drawings, exemplary embodiments are described. The drawings are schematic illustrations of idealized example embodiments. Thus, the example embodiments discussed herein should not be construed as limited to the particular illustrations presented herein, rather these example embodiments can include deviations and differ from the illustrations presented herein.

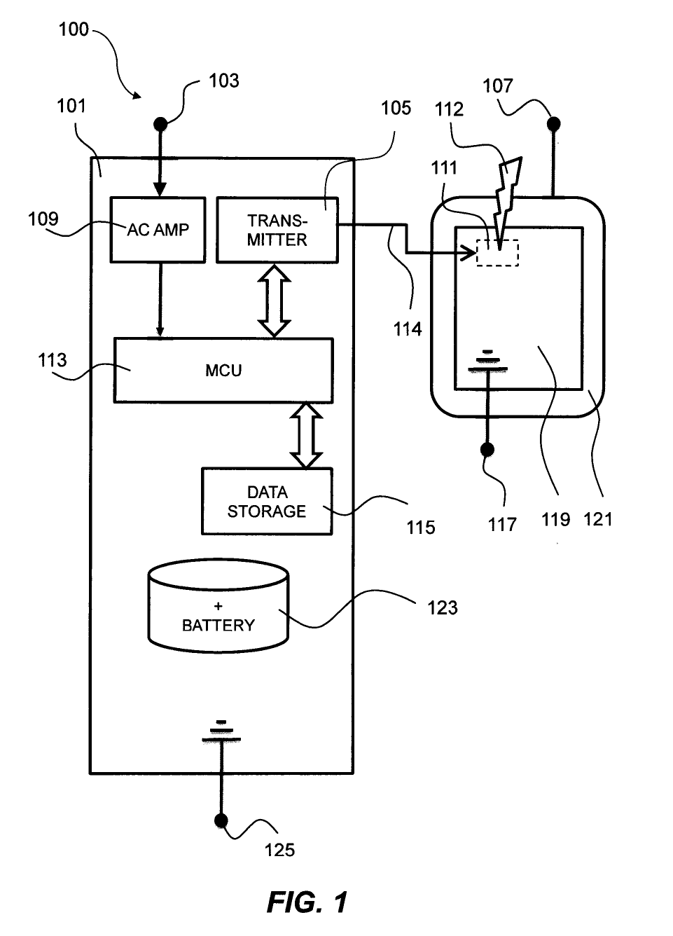

FIG. 1 shows a block diagram representing an example apparatus 100 for heart activity monitoring apparatus also called heart monitoring system connected to a host via wireless protocol such as Bluetooth, ANT+, ZigBee or a proprietary wireless protocol for the purpose of measuring and recording activity characteristics of a human heart.

As shown on FIG. 1, the heart monitoring system 100 is comprised of two sensors. The first sensor outlined by a sensor 101 can be a standalone device or be integrated into a wearable device and called a supplier sensor. The second sensor outlined by a rectangle 119 can be integrated into a wearable device 121 such as a watch and called a consumer sensor. The supplier sensor 101 can wirelessly communicate with a consumer sensor 119 via transmitters 105 and 111 using one or many wireless protocols 114 such as Bluetooth, ANT+, ZigBee or a customized wireless protocol.

The sensor 101 consists of the following logical elements: an electrode 103 connected to an input of an alternating current voltage amplifier (AC AMP) 109. The output of the AC AMP 109 is an amplified voltage potential measured between the electrode 103 attached to skin and a ground or common negative point 125. The output of the AC AMP 109 is connected to an analogue input of microcontroller (MCU) 113. MCU 113 processes the voltage potential into a digital format and stores it in a data storage 115 like a flash memory. At predefined time intervals, the MCU 113 sends the data, stored in the data storage 115, to the receiving consumer sensor 119 via the transmitter 105 and received via transmitter 111 as an array of measured data. The transmission time interval is set by the consumer sensor 119 at the time of establishing wireless connection 114 with the supplier sensor 101 and can be changed at any time during connection.

Modern integrated circuits (IC) can have MCU 113, AC AMP 109, transmitter 105, and data storage 115 integrated into a single IC (commonly called a microchip). A battery 123 provides power for the sensor 101. It can be a rechargeable or a single use (non-rechargeable) battery. The contact point 125 is connected to a ground or common negative point of the sensor 101.

The consumer sensor outlined by a rectangle 119 has the same logical components as described for the supplier sensor outlined by a sensor 101. It also has an electrode 107 similar to the electrode 103 and the ground or common negative connector 117 similar to connector 125. The consumer sensor 119 has wireless transmitter 111 with the same wireless protocol as the transmitter 105. Both transmitters 105 and 111 can wirelessly communicate simultaneously with other devices such as a smartphone via wireless protocols such as Bluetooth, ANT+, ZigBee, Wi-Fi or a customized wireless protocol.

The sensor outlined by a rectangle 119 can be integrated into a wearable device 121 such as a watch, an arm band, glasses, goggles, a headphone, or any other device capable of receiving wireless signals, processing, displaying, storing or rebroadcasting to another device. When integrated with a wearable device, the sensor 119 can share the physical components of the wearable device such as AC AMP, MCU, data storage, transmitter and the battery.

The decision of which sensor becomes a supplier or a consumer is negotiated at the time of establishing a connection between both sensors 119. A user can also select which sensor will be operating as a consumer sensor. The sensor configured as a consumer sensor nominates the other connected sensor as supplier. A consumer sensor can be connected to one or more supplier sensors. Typically, a sensor integrated into a wearable device which has an ability to interface with the user via touch, voice, or gesture commands is usually defined as the consumer sensor.

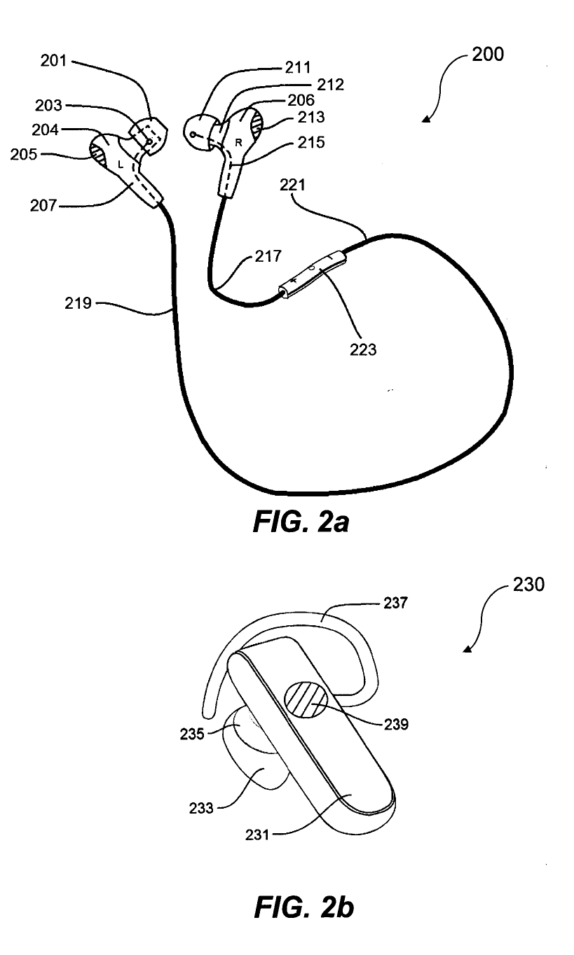

Other design variations with the heart monitoring sensor attached to or integrated into wearable accessories or devices with electrodes connected to skin using the wearable's existing points of contact with the skin can be utilized using the disclosure. The above examples as presented in FIG. 2 through FIG. 9 are not limited to only the described wearable devices or accessories and electrode connection options. In the spirit of this disclosure, any wearable device or accessory can be enhanced with the disclosed heart monitoring sensor where the electrode is connected using specific features of a wearable device, equipment, or clothing.

### Examples Illustrating the Use of Headset

FIG. 2a is a view illustrating one embodiment of the invented V-potential sensor integrated with a wireless stereo headset 200. The headset consists of the left headphone assembly 204, the right headphone assembly 206, and the headset controller 223. The headset controller 223 contains a battery, a digital signal process for audio playback, a wireless transmitter and audio control functions such as volume controls marked “+” and “−”, and a multifunctional programmable button in the middle. These features are common in traditional headsets. In addition, the headset controller 223 contains the invented V-potential sensor whose components are logically illustrated inside the sensor enclosure 101 as shown on FIG. 1. Some components such as the battery, wireless transmitter, data storage, and MCU can be used for dual purpose: to control headset operations and the invented skin voltage potential sensor operations.

An in-ear insert 201 is made from an electrically conductive polymer such as latex, silicone, or synthetic rubber with additives allowing electrical current to pass through the material with low resistance. The in-ear insert 201 can also be made from a soft or spongy material with electrical metal wires woven into the material. The purpose for the electrically conductive in-ear insert 201 is to fit gently into the ear and establish a reliable electrical contact with the skin. The in-ear insert 201 is replaceable and fitted over a headphone assembly base 203. The base 203 for the in-ear inserts 201 is made from a metal or electrically conductive material and electrically connected to the in-ear insert 203.

The base 203 is connected to a wire 207 which is then fitted through the headphone assembly 204 into a cable 219. A typical audio cable contains a wire for the headphone audio signal and a ground or common negative wire. In addition, the wire 207 is added into the cable 219 for V-potential signal measurement and connected in the headset controller 223 as an electrode. The ground or common negative wire is electrically connected to a conductive pad 205 located on the outside surface of the left headphone assembly 204.

The in-ear insert 201 is electrically connected to the headset controller 223 and logically represented as an electrode 103 on FIG. 1. The conductive pad 205 is connected to the headset controller 223 and logically represented as a contact 125 on FIG. 1. The headset controller 223 contains components of the V-potential sensor as logically illustrated by a sensor 101 on FIG. 1 and comprises the first V-potential supplier sensor.

In another embodiment, in addition to the left earphone assembly 204, the right earphone assembly 206 can also have an electrically conductive in-ear insert 211 which is fitted over an electrically conductive base 212. The base 212 is connected via a wire 215 to the headset controller 223. The headphone assembly 206 has a conductive pad 213 connected to the ground or a common negative wire. The wire 215 is a part of the cable 217, which also contains wires for the right headphone audio signal and a ground or common negative wire. The right earphone assembly 206 comprises the second V-potential supplier sensor. The in-ear insert 211 is electrically connected to the headset controller 223 and logically represented as an electrode 103 on FIG. 1. The conductive pad 213 is connected to the headset controller 223 and logically represented as a contact 125 on FIG. 1. The headset controller 223 contains components of the second V-potential sensor as logically illustrated by a sensor 101 on FIG. 1.

It is apparent to a person with ordinary skills that the first and the second V-potential supplier sensors integrated into one headset share a common enclosure. In this configuration, both sensors may also share logical components such as MCU 113, transmitter 105, data storage 115, and the battery 123 illustrated on FIG. 1.

In yet another embodiment as illustrated in FIG. 2b, the invented V-potential sensor is integrated with a wireless mono headset 230. An in-ear insert 233 is made from an electrically conductive polymer such as latex, silicone, or synthetic rubber with additives allowing electrical current to pass through the material with low resistance. The insert 233 is fitted over a metal or electrically conductive base 235. The base 235 can be electrically connected to an optional over-the-ear holder 237, which is made from an electrically conductive polymer or a metal. The over-the-ear holder 237 establishes an additional electrical contact with the skin behind the ear for more reliable V-potential measurement. A headset controller located inside an enclosure 231 contains the components logically illustrated inside the sensor enclosure 101 on FIG. 1. Some components such as the battery, wireless transmitter, data storage, and MCU can be used for dual purpose: control the headset operations and the skin V-potential monitoring.

The base 235 is electrically connected to the enclosure 231 and logically illustrated as electrode 103 on FIG. 1. The ground or common negative wire is electrically connected to a conductive pad 239 located on the surface of the headset enclosure 231. The conductive pad 239 is logically illustrated as ground contact 125 on FIG. 1. The size and shape of the conductive pad 239 can vary based on shape and form of the headset enclosure 231. The pad 239 shall be easily accessible when the headset is worn in the ear but not interfere with regular operations of the headset.

Both headsets 200 and 230 can be implemented in different shapes and forms connecting to one or both ears. Regardless of the shape and form of the headset and the number of earphones, the principle of this disclosure remains the same: one or both in-ear inserts provide electrical contacts with the skin of the ear for the purpose of measuring the skin V-potential signal.

The description of the headsets is focused on the skin V-potential sensor and omits common components found in most headphones for the purpose of transmitting audio signals. The traditional headset controller may have similar components as illustrated on FIG. 1 such as MCU 113, AC AMP 109, transmitter 105, and data storage 115 which could be integrated into a single IC. A common set of components can be used for both functions: audio playback and skin V-potential monitoring. The entire system with audio and the invented skin V-potential sensor can share a single battery and be integrated in one enclosure.

### Examples Illustrating the Use of a Watch, Pendulum, or a Button

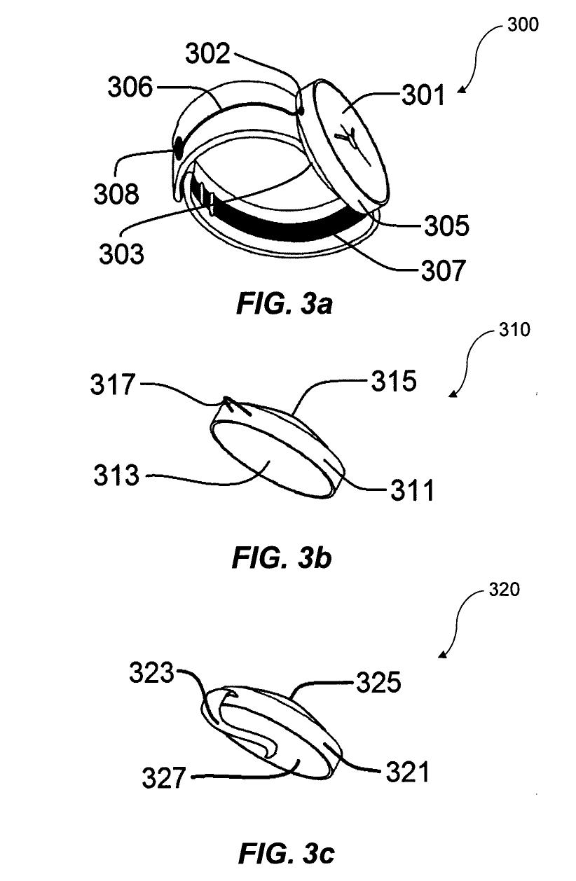

FIG. 3a illustrates one embodiment of the invented V-potential sensor integrated with a watch 300. This implementation is logically illustrated as a consumer sensor 119 on FIG. 1. A back pad 303 of the watch 300 is made from an electrically conductive material or has an electrically conductive pad and is logically represented as the electrode 107 on FIG. 1. A wrist band can also have an electrically conductive wire 307 made from a polymer or a metal. The entire wrist band can be made from a metal or a conductive polymer and be electrically connected to the pad 303. The frame of the watch 300 is made from an electrically conductive material or has an electrically conductive pad which is logically represented as ground contact 117 on FIG. 1. Alternatively, the ground contact can be electrically connected to a wire integrated into a band or a conductive wire 306 via an electrical connector 302. The conductive wire 306 connects to a conductive pad 308 used for synchronizing a ground or common negative point. The frame 305 and the back pad 303 of the watch 303 are electrically isolated from each other. The wire 306 and the wire 307 are also electrically isolated from each other, located on different sides of the watch band.

FIG. 3b illustrates one embodiment of the invented V-potential sensor integrated in the enclosure for wearing on a necklace like a pendulum 310. The V-potential sensor is integrated into the pendulum as a stand-alone sensor. The back pad of the pendulum 315 and the frame of the pendulum 311 are made from an electrically conductive material. Both the frame 311 and the back pad 315 are electrically connected or made from a solid metal or a conductive polymer and are logically represented as the electrode 103 on FIG. 1. The hook 317 for hanging the pendulum onto a chain or necklace is also made from an electrically conductive material and electrically connected to the frame 311. The face pad 313 of the pendulum is made from an electrically conductive material and logically represented as the electrode 125 on FIG. 1. The face pad 313 of the pendulum is electrically isolated from the frame 311 and the back pad 315. The face pad 313 can also have a display like a watch or LED indicators.

FIG. 3c illustrates another embodiment of the invented V-potential sensor integrated in a button-like enclosure 320. It can be clipped to or worn on a piece of clothing. The V-potential sensor can be integrated into a button-like housing 321. The back pad 325 of the button is made from an electrically conductive material and is logically represented as the electrode 103 on FIG. 1. The face pad 327 of the button is made from an electrically conductive material and is logically represented as the electrode 125 on FIG. 1. The housing 321, the face pad 327, and the back pad 325 of the button are electrically isolated from each other. A hook 323 for attaching the button onto a piece of clothing can also be electrically connected to the face pad 327 and also plays the role of the electrode 125 as illustrated on FIG. 1.

The examples of the V-potential sensor integration described in FIGS. 2a, 2b, 3a, 3b, and 3c will be further illustrated as a system of V-potential sensors consisting of at least one consumer sensor and one or many supplier sensors.

### Examples Illustrating the Use of V-Potential Sensors as a System

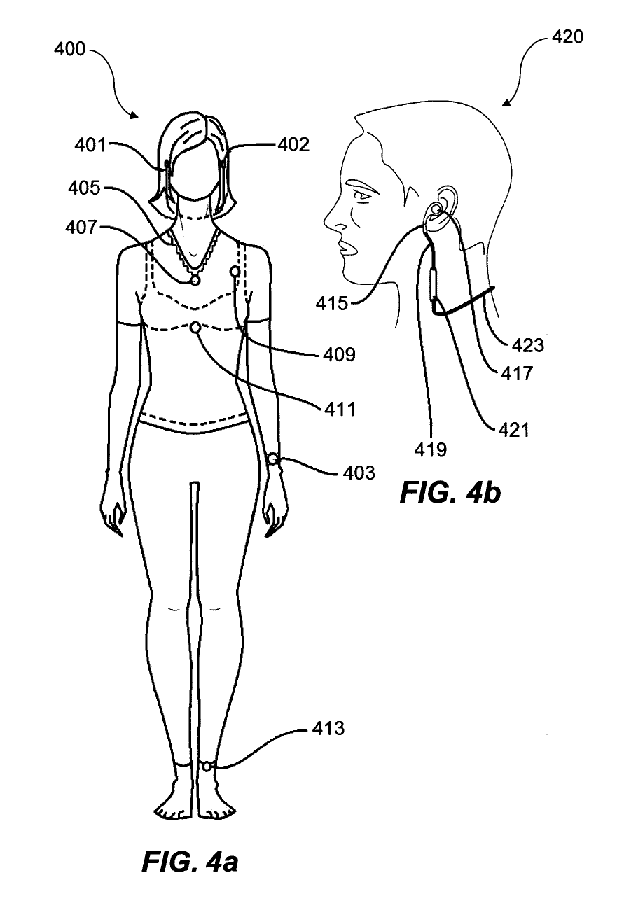

FIG. 4a illustrates different options for wearing V-potential sensors on a human body as part of a heart monitoring sensory system 400. FIG. 4b illustrates one embodiment of the invented V-potential sensor integrated into a wireless stereo headset 420. The left in-ear phone establishes the first electrical contact 402. FIG. 4b shows the side view of a human head with the V-potential sensor integrated into a wireless stereo headset 420. A headset controller 421 is connected to a left earphone 415 via a cable 419 and to a right earphone via a cable 423. The left earphone 415 has a pad 417 connected to a ground or a common negative point. The pad 417 is also illustrated on FIG. 2a as the pad 205. The headset integrates the first V-potential sensor.

The second V-potential sensor is integrated into a watch 403 as illustrated on FIG. 4a. The V-potential sensor in the watch 403 is configured as a consumer sensor by the user. When the consumer sensor integrated into the watch 403 establishes a wireless connection to the first sensor integrated into the headset 402, it instructs the first sensor to act as a supplier sensor and sets the sampling and communication frequency (for example, 20 Hz sampling frequency and 2 Hz communication frequency).

The supplier sensor starts sampling skin V-potential at the defined 20 Hz sampling frequency, stores data samples in its memory, and then transmits collected batches of data to the consumer sensor at a 2 Hz communication frequency.

The consumer sensor integrated in a watch 403 stores received batches of data from the supplier sensor 402 in its internal memory and then compares the data with its own measurement of skin V-potential. The consumer sensor's MCU analyzes both data sets using the ECG or EKG method for heart activity detection and calculates heart activity characteristics such as the heart rate or heart beats per minute, heart variability rate, and heart rhythm.

Each sensor contains location identification such as in-ear location for the sensor integrated into the headset 402 and a wrist location for the sensor integrated into the watch 403. This location identification helps to fine tune an algorithm for detecting heart activity using the ECG method.

As part of operating the invented heart activity sensory system, the ground or common negative point shall be synchronized periodically for both consumer and supplier sensors. This is achieved by touching the ground pad on the watch 403 to the ground pad of a headset. The user can lift the hand with a watch to the ear and connect ground pads of the watch to the ground pad of the headset. A tactile or audible feedback confirms that the ground is synchronized for both sensors. The ground pad of the watch is shown as 308 on FIG. 3a. The ground pad of the headset is shown as 205 on FIGS. 2a and 417 on FIG. 4b. The ground synchronization is needed the first time after the sensors are wirelessly connected. The consumer sensor alerts the user to synchronize ground with a tactile, audible, visual, or voice feedback. During operation, the consumer sensor can detect if a new ground synchronization is required based on the quality of the signal received from the supplier sensor.

A calibration can also be performed to confirm that the invented sensory system has reliable contacts between electrodes and the skin where they are located and it can reliably measure heart activity using the traditional ECG method. The user shall keep both ground pads in contact until the consumer sensor collects enough data from the supplier sensor to calculate heart activity characteristics. A tactile, audible, visual, or voice feedback confirms that the calibration is completed.

In another embodiment of the invented heart activity sensory system as illustrated on FIG. 4a, the first skin V-potential sensor is integrated with a necklace pendulum 407 hanging on a neck chain 405. The pendulum 407 is illustrated in detail on FIG. 3b. The back pad of the pendulum 407 establishes a contact with the skin on a chest. An additional contact can be provided from a metal neck chain or electrically conductive necklace connected through the hook 317 of the pendulum. The chain or necklace contacts the skin around the neck of the user.

The second skin voltage potential sensor is integrated into a watch 403. The sensor in the watch 403 is configured as a consumer sensor. Upon connecting wirelessly, the consumer sensor in the watch 403 instructs the first sensor in the pendulum 407 to act as a supplier sensor and sets the sampling and communication frequency. The operation of both sensors is similar to the operation of the watch and the headset combination described above.

In yet another embodiment of the invented heart activity sensory system, as illustrated on FIG. 4a, the first skin V-potential sensor is integrated with a button sensor attached to a bra 411. The button sensor is illustrated in detail on FIG. 3c. The back pad of the button sensor 411 establishes a contact with the skin under the bra. The second skin V-potential sensor is integrated into a watch 403 (also illustrated in detail on FIG. 3a). The sensor in the watch 403 is configured as a consumer sensor. Upon connecting wirelessly, the consumer sensor instructs the first sensor in a button to act as a supplier sensor and sets the sampling and communication frequency. The operation of both sensors is similar to the operation of the watch and the headset combination.

Synchronizing the common ground is done by touching common ground electrodes of both consumer and supplier sensors. This can also be done with a help of a wire or a conductive cord in cases when one or both sensors are not exposed or conveniently located for an easy reach.

It is clear to a person with ordinary skills in the art that consumer and supplier sensors can be integrated into different devices and attached to different places on a human body. Other combinations are possible in addition to the described embodiments. For example, a supplier sensor can be a button 409 attached to a bra strap and a consumer sensor in an earphone 401. In yet another embodiment, a supplier sensor can be integrated in a button 413 and attached to leg tights. The consumer sensor is integrated in a watch 403. Other wearable devices can be used to integrate skin V-potential sensors.

### Examples Illustrating the Use of Glasses or Googles

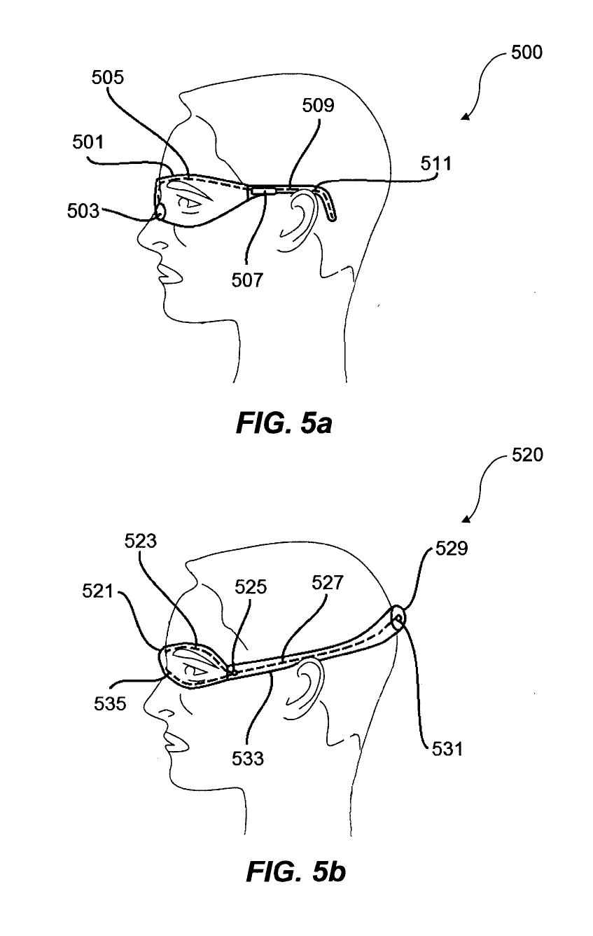

FIG. 5a illustrates one embodiment of the invented V-potential sensor integrated with glasses. In one embodiment, the invented V-potential sensor is integrated into glasses 500. The skin contact is established at nose pads 503. The nose pads 503 are made from an electrically conductive polymer. The nose pads 503 are connected to a sensor assembly 507 via an electrical cable 505 fitted in the frame of the glasses 501 or via a frame of the glasses 501 when the frame is made from a conductive material like a metal or conductive polymer. An electrode cable 511 can be installed on the temple frame 509 of the glasses in such a way that it establishes an additional contact with skin around the temple and behind the ear. Alternatively, the entire temple frame 509 can be made from a conductive material like a metal or a conductive polymer. The sensor assembly 507 is logically represented as 101 on FIG. 1. The electrode 103 is combined from nose pads 503 and temple frame 511. The common ground electrode 125 as shown on FIG. 1 can be located on the outer surface of the sensor enclosure 507.

In one embodiment, the V-potential sensor 507 can be configured as a supplier sensor when connected to a consumer sensor integrated into a watch. In another embodiment, the sensor 507 can be a consumer sensor and connected with supplier sensors integrated into other wearable devices or attached to different parts of the body.

FIG. 5b illustrates one embodiment of the invented V-potential sensor integrated with goggles 520. The skin contact is established with wires 523 and 535 connected at a splitter 525. The contact wires 523 and 535 are made from an electrically conductive material like a conductive polymer and embedded into the eye pads of the googles. The goggle's eye pads with conductive wires 523 and 535 are pressed against the skin around the eyes and establish a reliable electrical connection with the skin. The splitter 525 is connected via an electrical cable 527 located inside a band 533 to skin voltage potential sensor 529. The sensor 529 can be attached to goggles 520 with an electrically conductive snap button 531, which is connected to the cable 527. The sensor 529 is logically represented on FIG. 1 as the sensor 101. The electrode 103 is attached to the goggles via a connector 531. The common ground electrode 125 is positioned on the outer surface of the sensor 529.

In one embodiment, the V-potential sensor 529 can be configured as a supplier sensor when connected to a consumer sensor. In another embodiment, the sensor 529 can be a consumer sensor and connected with supplier sensors integrated into other wearable devices or attached to different parts of the body.

The ground synchronization and the operation of both sensors integrated into glasses or goggles are similar to the sensors integrated into the headset 402 and the watch 403 as described in FIG. 4.

### Examples Illustrating the Use of a Hat

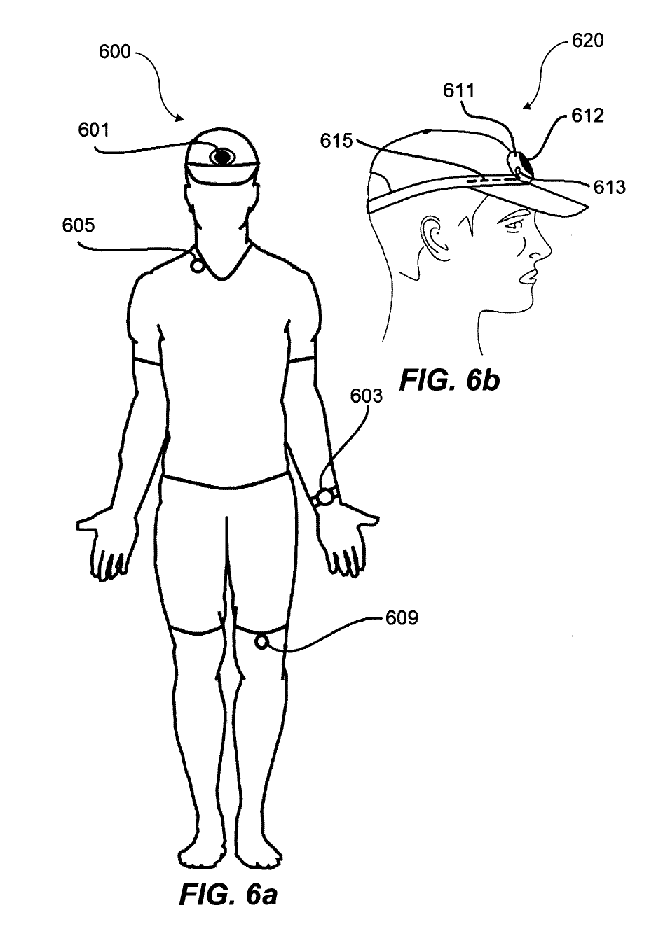

FIG. 6a illustrates another embodiment for wearing an invented heart monitoring sensory system 600. The first V-potential sensor 601 is attached to a hat. The side view on FIG. 6b illustrates how the V-potential sensor could be attached to a hat 620. The V-potential sensor 612 is clipped onto a hat with a connector 613, which could be a metal snap button or other connector capable of conducting electrical current. The connector 613 is attached to an electrically conductive wire 615, which is integrated into the inner lining of the hat and establishes a contact with skin on the forehead. The wire 615 is made from an electrically conductive polymer or a metal wire woven into the fabric of the lining.

The connector 613 is logically represented as an electrode connector 103 on FIG. 1. The pad 612 on the outer body 611 of the sensor is made from a conductive material and connected to the ground or common negative point, which is logically represented as a contact 125 on FIG. 1.

The V-potential sensor 601 illustrated on FIG. 6a is wirelessly connected to a consumer sensor integrated into a watch 603. The sensor 601 is configured to act as a supplier sensor after establishing connection with a consumer sensor. The ground synchronization and the operation of both sensors 601 and 603 are similar to the sensors integrated into a headset 402 and a watch 403 as illustrated on FIG. 4a.

The V-potential potential sensor 601 can also be attached to other head accessories such as a helmet, headband, bandana, or any other wearable device that can be worn on a head and contact the skin of the forehead. Regardless of the connector type, the principal of the invented sensor remains the same: an electrode from the sensor is connected to skin on the forehead with the help of a hat or other head wearable accessory.

In yet another embodiment of the invented heart activity sensory system, as illustrated on FIG. 6a, the first V-potential potential sensor is integrated with a button attached to a top garment 605, or to a bottom garment 609 (like shorts or leg tights). The button sensor is illustrated in detail on FIG. 3c. The back pad of the button sensor establishes a contact with the skin on the chest 605 or the leg 609 (also logically illustrated as the electrode 103 on FIG. 1. The hook 323 on FIG. 3c of the button sensor provides a ground or common negative contact 125 as shown on FIG. 1.

The second skin voltage potential sensor is integrated into a watch 603 (also illustrated in detail on FIG. 3a). The sensor in the watch 603 is configured as a consumer sensor. Upon connecting wirelessly, the consumer sensor integrated into a watch 603 instructs the button sensor 605 or 609 to act as a supplier sensor and sets the sampling and communication frequency. The ground synchronization and the operation of V-potential supplier sensors 605 or 609 with the consumer sensor integrated into a watch 603 are similar to the sensors integrated into a button and a watch illustrated on FIG. 4.

Other design variation and placement of the invented skin voltage potential sensors attached to or integrated into wearable accessories or devices with electrodes connected to skin using wearable's existing points of contact with the skin can be utilized using the disclosure. The above examples presented on FIG. 2 through FIG. 6 are not limited to only the described wearable devices or accessories and electrode connection options.

### Examples Illustrating the ECG Signal Measured at Different Parts of the Body

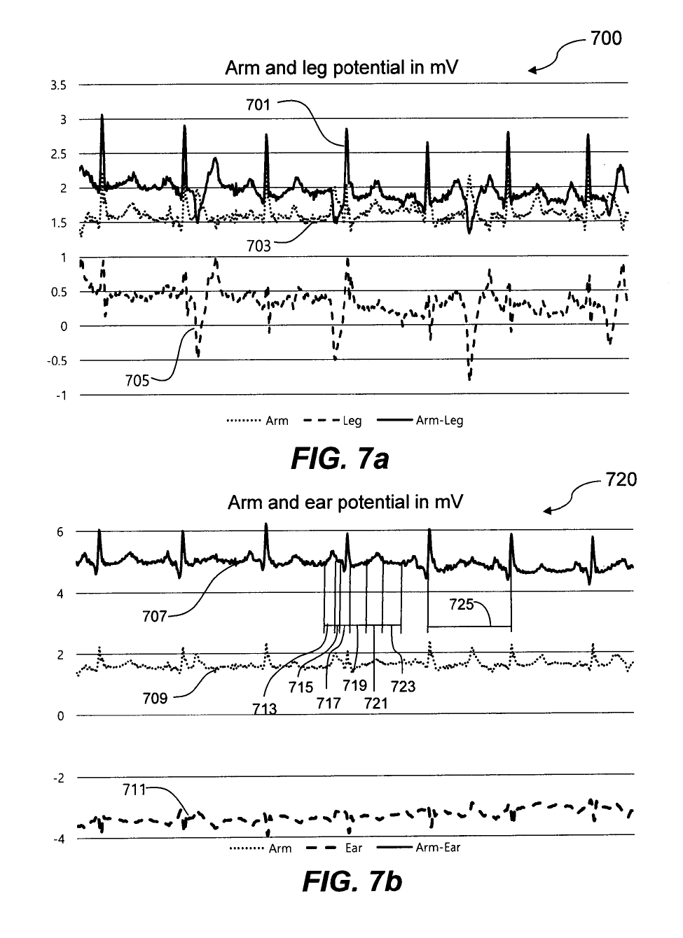

FIG. 7a illustrates the voltage potential recorded with the first V-potential sensor attached to a leg 609 as shown on FIG. 6a and a second voltage potential sensor mounted into a wrist wearable device like a watch or a wrist band 603 as shown on FIG. 6a. A dotted line 703 represents voltage potential in millivolts (mV) recorded by the V-potential sensor placed on the wrist as a watch. A dashed line 705 represents voltage potential in millivolts (mV) recorded by the V-potential sensor placed on the leg, just above the knee on the back side of the leg. A solid line 701 represents voltage differential between arm and leg measurement.

FIG. 7b illustrates the voltage potential recorded with the first V-potential sensor inserted into the left ear 402 as shown on FIG. 4a and a second V-potential sensor mounted into a wrist wearable device like a watch 403 as shown on FIG. 4a. A dotted line 709 represents voltage potential in millivolts (mV) recorded by the V-potential sensor placed on a wrist. A dashed line 711 represents voltage potential in millivolts (mV) recorded by the V-potential sensor placed in the ear 402 as shown on FIG. 4a. A solid line 707 represents voltage differential between arm and ear measurement.

It is apparent that the voltage potential measured in a single location 703 and 705 as illustrated on FIG. 7a, and 709 and 711 as illustrated on FIG. 7b does not provide a clear ECG signal for heart activity monitoring. The differential voltage potential 701 on FIGS. 7a and 707 on FIG. 7b represent clear ECG waveform where 713 is P wave, 715 is PR segment, 717 is QRS complex, 719 is ST segment, 721 is T wave, and 723 is U wave. Using higher amplification and standard signal filtering methods can provide much smoother ECG signal 707. The heart rate can be clearly seen as an interval between QRS peaks 725.

Recording and interpretation of ECG signal 701 can be conducted on the consumer sensor as described above and translated into a heart rate and other information deemed to be useful for heart monitoring by a particular implementation of a wearable device with a second sensor.

### Example Method for Supplier Sensor Operation

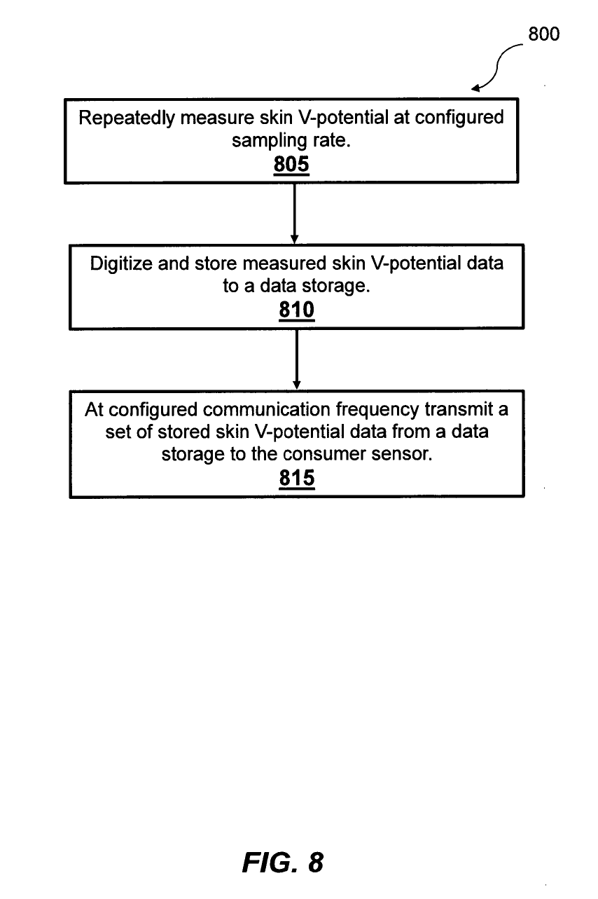

FIG. 8 is a process flow diagram showing a method 800 for V-potential supplier sensor operation. Method 800 may be performed by processing logic that may comprise hardware (e.g., decision-making logic, dedicated logic, programmable logic, application-specific integrated circuit), software, or a combination of both. In one example embodiment, the processing logic refers to sensor 101. Below recited operations of method 800 may be implemented in an order different than described and shown in the figure. Moreover, method 800 may have additional operations not shown herein, but which can be evident for those skilled in the art from the present disclosure. Method 800 may also have fewer operations than outlined below and shown in FIG. 8.

Method 800 commences at operation 805 when the supplier sensor repeatedly measures skin V-potential at configured sampling rate. At operation 810 the MCU 113 processes the skin V-potential signal measured by the supplier sensor using electrode 103 and converts it to a digital format. Then MCU 113 stores the converted data in a data storage 115. At operation 815 MCU 113 transmits a set of skin V-potential data, stored in a data storage 115 as a batch or a stream to the consumer sensor via transmitter 105. The data transmission event occurs at previously configured communication frequency. Then the method 800 continues from operation 805.

### Example Method for Consumer Sensor Operation

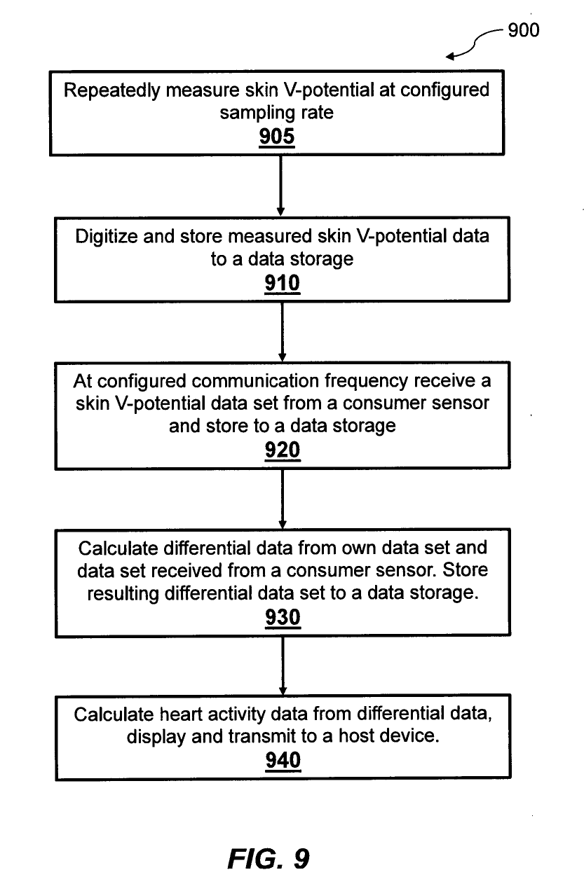

FIG. 9 is a process flow diagram showing a method 900 for V-potential consumer sensor operation. Method 900 may be performed by processing logic that may comprise hardware (e.g., decision-making logic, dedicated logic, programmable logic, application-specific integrated circuit), software, or a combination of both. In one example embodiment, the processing logic refers to sensor 119 integrated into a wearable device 121 such as a watch. Below recited operations of method 900 may be implemented in an order different than described and shown in the figure. Moreover, method 900 may have additional operations not shown herein, but which can be evident for those skilled in the art from the present disclosure. Method 900 may also have fewer operations than outlined below and shown in FIG. 9.

Method 900 commences at operation 905 when the consumer sensor repeatedly measures skin V-potential at configured sampling rate. At operation 910 the MCU processes the skin V-potential signal measured by the consumer sensor using electrode 107 and converts it to a digital format. Then MCU stores the converted data in a data storage. At operation 920 the consumer sensor receives a batch of data from a supplier sensor containing V-potential data set measured by the supplier sensor. The data transmission event occurs at previously configured communication frequency. At operation 930 consumer sensor's MCU calculates differential data from its own V-potential data set and V-potential data set received from a consumer sensor. The resulting data set comprises differential V-potential data illustrated as 701 and 707. The differential V-potential data set is then recorded to a data storage. At operation 940 consumer sensor's MCU calculates heart activity data from differential V-potential data set, displays and notifies the user by other means. The consumer sensor can further transmit the heart activity data to a host device like a smartphone. Then the method 900 continues repeatedly measure V-potential signal from operation 905.

### Examples Illustrating the Use of Multiple Supplier Sensors

In some cases, it is advantageous to have multiple V-potential supplier sensors connected to one V-potential consumer sensor. Multiple V-potential sensors provide more reliable ECG measurements. As illustrated on FIG. 4a, the user can wear a stereo headset with earphones in the right 401 and the left 402 ears. As illustrated in FIG. 2a, the headset 200 can have two independent V-potential sensors integrated in each of the left and right earphones. As illustrated on FIG. 4a, the V-potential sensor in right ear 401 measures electrical signal on the skin in the right ear and the V-potential sensor in left ear 402 measures electrical signal on the skin in the left ear. Both sensors share the same ground or a common negative point since they are integrated into one headset device.

The V-potential sensor in the watch 403 is configured as a consumer sensor by the user. When the consumer sensor in the watch 403 establishes a wireless connection to the sensor integrated into the right earphone 401, it instructs the sensor 401 to act as the first supplier sensor and sets the sampling and communication frequency. When the consumer sensor in the watch 403 establishes a wireless connection to the sensor integrated into the left earphone 402, it instructs the sensor 402 to act as the second supplier sensor and sets the sampling and communication frequency.

Both supplier sensors start sampling skin V-potential at defined sampling frequency, store data samples in memory, and then transmit collected batches of data to the consumer sensor at a defined communication frequency.

The consumer sensor integrated into a watch 403 stores received batches of data from both supplier sensors 401 and 402 in its internal memory and then compares the data with its own measurement of skin V-potential. The consumer sensor's MCU analyzes data from all 3 points on the skin using the ECG or EKG method for heart activity detection and calculates heart activity characteristics such as the heart rate or heart beats per minute, heart variability rate, and heart rhythm.

The number of consumer sensors is not limited to one or two but rather is limited by practical usage and desired quality and reliability of ECG data. Other sensors can also be connected to a consumer sensor 403 as supplier sensors. All supplier sensors can be synchronized for the first time as was already described for each pair of sensors.

Other design variations with the skin V-potential monitoring sensors attached to or integrated into wearable accessories or devices with electrodes connected to skin using wearable's existing points of contact with the skin can be utilized using the disclosure. The above examples presented in FIG. 1 through FIG. 9 are not limited to only the described wearable devices or accessories and electrode connection options. In the spirit of this disclosure, many wearable devices or accessories can be enhanced with the disclosed skin voltage potential monitoring sensor where the first sensor is connected via an electrode to the skin using specific features of a wearable device, equipment, or clothing for a tight contact with the skin. The second sensor is connected via an electrode to another point on the skin using existing wearable devices, equipment, or clothing. The first sensor is configured as a supplier and wirelessly transmits skin voltage potential data to the second sensor configured as a consumer. The consumer sensor uses data from the supplier sensor together with its own skin voltage potential measurement, calculates a voltage difference, and applies the ECG method to calculate heart activity characteristics. The resulting heart activity characteristics can be wirelessly transmitted to another device and shown on a display, played back with voice or light indicators, or communicated via tactile cues to the human.

Thus, an apparatus for heart activity monitoring has been described with different mounting options to wearable accessories. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

1. A system for heart activity monitoring, the system comprising:
at least one first sensor positioned within at least one first area of a body of a user and integrated into a first accessory worn by the user, the at least one first sensor being configured to sense at least one electrical signal from skin of the user within the at least one first area of the body of the user, the at least one electrical signal being indicative of heart activity of the user; and
a second sensor positioned within a second area of the body of the user and integrated into a second accessory worn by the user, the second sensor being configured to:
sense a second electrical signal from the skin of the user within the second area of the body of the user, the second electrical signal being indicative of the heart activity of the user and being sensed simultaneously with the at least one electrical signal;
wirelessly receive the at least one electrical signal from the at least one first sensor; and
perform a comparative analysis of the at least one electrical signal and the second electrical signal to obtain heart activity data.

2. The system of claim 1, wherein:
the at least one first sensor includes:
a first common negative point;
a first electrode configured to sense the at least one electrical signal;
a first voltage amplifier configured to amplify a voltage potential between the first electrode and the first common negative point;
a first microcontroller configured to convert the first voltage potential to first digital data;
a first data storage configured to store the first digital data; and
a first wireless transmitter configured to wirelessly transmit the first digital data;
the second sensor includes:
a second common negative point;
a second electrode configured to sense the second electrical signal;
a second voltage amplifier configured to amplify a second voltage potential between the second electrode and the second common negative point;
a second microcontroller configured to convert the second voltage potential to second digital data;
a second wireless transmitter configured to wirelessly receive the first digital data; and
a second data storage configured to store the first digital data and the second digital data; and wherein:
the second microcontroller is further configured to calculate, based on the first digital data and the second digital data, heart activity data; and
the second wireless transmitter is further configured to wirelessly transmit the heart activity data.

3. The system of claim 2, wherein the at least one first sensor and the second sensor are periodically synchronized by connecting the first common negative point and the second common negative point.

4. The system of claim 1, wherein the first accessory and the second accessory are configured to have a direct contact with skin of the user.

5. The system of claim 1, wherein at least one of the first accessory and the second accessory includes at least one of the following: a hat, a cap, a helmet, a bandana, a head cover, glasses, skiing googles, swimming googles, protective googles, a watch, an armband, a wrist band, a leg band, a button, a brooch, and an ear clip.

6. The system of claim 2, wherein:
the first accessory includes an elastic insert; and
the first electrode is integrated with the elastic insert.

7. The system of claim 2, wherein:
the first accessory includes a metal insert; and
the first electrode is integrated with the metal insert.

8. The system of claim 2, wherein:
the first accessory includes one of the following: a hat, a cap, a helmet, a bandana, and a head cover; and
the first electrode establishes a direct contact with forehead skin of the user.

9. The system of claim 2, wherein:
the first accessory includes one of glasses or eyewear; and
the first electrode establishes a direct contact with the skin on a nose of the user.

10. The system of claim 2, wherein:
the first accessory includes one of the following: skiing googles, swimming googles, protective googles, or another type of goggles; and
the first electrode establishes a direct contact with the skin around eyes of the user.

11. The system of claim 2, wherein:
the at least one first sensor integrated with an earpiece, the earpiece including an earbud with an elastic in-ear insert; and
the first electrode is integrated with the elastic in-ear insert.

12. The system of claim 11, wherein the elastic in-ear insert of the earbud includes an electrically conducting material.

13. The system of claim 2, wherein the first electrode establishes a direct contact with the skin of a temple, above the ears, or behind the ears of the user.

14. The system of claim 1, wherein the heart activity data include one or more of the following: a heart rate, heart beats per minute, a heart variability rate, a heart rhythm, and an electrocardiogram represented in digital form.

15. The system of claim 1, further comprising a host device configured to wirelessly receive the heart activity data; and
wherein the second sensor is further configured to wirelessly transmit the heart activity data to the host device.

16. A method for heart activity monitoring, the method comprising:
sensing, by at least one first sensor positioned within at least one first area of a body of a user and integrated with a first accessory worn by the user, at least one electrical signal from skin of the user within the at least one first area of the body of the user, the at least one electrical signal being indicative of heart activity of the user;
sensing, by a second sensor positioned within a second area of the body of the user and integrated with a second accessory worn by the user, a second electrical signal from the skin of the user within the second area of the body of the user, the second electrical signal being indicative of the heart activity of the user and being sensed simultaneously with the at least one electrical signal;
wirelessly receiving, by the second sensor, the at least one electrical signal;
performing, by the second sensor, a comparative analysis of the at least one electrical signal and the second electrical signal to obtain heart activity data; and
wirelessly transmitting, by the second sensor, the heart activity data to a host device configured to wirelessly receive the heart activity data.

17. The method of claim 16, wherein:
the at least one first sensor includes:
a first common negative point;
a first electrode configured to sense the at least one electrical signal;
a first voltage amplifier configured to amplify a first voltage potential between the first electrode and the first common negative point;
a first microcontroller configured to convert the first voltage potential to first digital data;
a first data storage configured to store the first digital data; and
a first wireless transmitter configured to wirelessly transmit the first digital data;
the second sensor includes:
a second common negative point;
a second electrode configured to sense the second electrical signal;
a second voltage amplifier configured to amplify a second voltage potential between the second electrode and the second common negative point;
a second microcontroller configured to convert the second voltage potential to second digital data;
a second wireless transmitter configured to wirelessly receive the first digital data;
a second data storage configured to store the first digital data and the second digital data; and
wherein:
the second microcontroller is further configured to calculate, based on the first digital data and the second digital data, the heart activity data; and
the second wireless transmitter is further configured to wirelessly transmit the heart activity data.

18. The method of claim 17, further comprising:
detecting, by the second sensor, that a synchronization of the at least one first sensor and the second sensor is required; and
in response to the detection, synchronizing the at least one first sensor and the second sensor by connecting the common negative point and the further common negative point.

19. The method of claim 17, wherein at least one of first accessory or the second accessory includes a hat, a cap, a helmet, a bandana, a head cover, glasses, skiing googles, swimming googles, protective googles, a watch, an armband, a wrist band, a leg band, a button, a brooch, and an ear clip.

20. The method of claim 17, wherein:
the first accessory includes one of glasses or eyewear; and
the first electrode establishes a direct contact with the skin on a nose of the user.

21. The method of claim 17, wherein the first electrode establishes a direct contact with the skin of a temple, above the ears, or behind the ears of the user.
